(19) United States
(12) Patent Application Publication
(10) Pub. No.: US 2020/0121665 A1
(43) Pub. Date: Apr. 23, 2020

(54) PPAR GAMMA MODULATORS FOR TREATING CYSTIC DISEASES

(71) Applicant: Indiana University Research and Technology Corporation, Indianapolis, IN (US)

(72) Inventor: Bonnie Blazer-Yost, Indianapolis, IN (US)

(21) Appl. No.: 16/580,186

(22) Filed: Sep. 24, 2019

Related U.S. Application Data
(63) Continuation of application No. 14/896,020, filed on Dec. 4, 2015, now abandoned.
(60) Provisional application No. 61/832,255, filed on Jun. 7, 2013.

(30) Foreign Application Priority Data
Jun. 7, 2013 (RU) 2013126422

(51) Int. Cl. A61K 31/4439; A61K 31/427; A61K 31/122; A61K 31/426; A61K 31/216; A61K 31/421; A61K 31/192

(52) U.S. Cl. CPC A61K 31/4439; A61K 31/427; A61K 31/122; A61K 31/192; A61K 31/216; A61K 31/421; A61K 31/426

(57) ABSTRACT

Compositions and methods are described herein for the treatment of cystic diseases using PPARγ modulators. In particular, compositions and methods are described herein for the treatment of cystic diseases using low doses of PPARγ modulators.

### CROSS REFERENCE TO RELATED APPLICATIONS

This application is a divisional application of U.S. application Ser. No. 14/896,020, filed on Dec. 4, 2015, which claims priority to International Publication No. WO 2014/197820, filed on Jun. 6, 2014, which claims priority to U.S. Provisional Patent Application No. 61/832,255 filed on Jun. 7, 2013 and RU Patent Application No. 2013126422 filed on Jun. 7, 2013, which are hereby expressly incorporated by reference in their entireties.

### STATEMENT OF GOVERNMENT SUPPORT

This invention was made with government support under RR025761 awarded by the National Institute of Health. The government has certain rights in the invention.

### TECHNICAL FIELD

The present disclosure described herein pertains to the treatment of cystic diseases using PPARγ modulators. In particular, the present disclosure described herein pertains to the treatment of cystic diseases using low doses of PPARγ modulators.

### BACKGROUND OF THE DISCLOSURE

Polycystic kidney diseases (PKDs) are genetic disorders that are characterized by the accumulation and growth of fluid-filled cysts in the kidney tubules and liver bile ducts. PKDs are often accompanied by fibrotic changes. It is normally diagnosed in adults with an incidence of 1:400 to 1:1000 (Harris et al., “Polycystic Kidney Disease,” Annu Rev Med, vol. 60, pp. 321-337, 2009). The disease progresses slowly and the fluid-filled cysts grow in size due to increased rates of proliferation and secretion. The expanding cysts compromise the normal kidney function and result in a decrease of renal function to the point of end-stage renal failure in midlife (Patel V. et al., “Advances in the pathogenesis and treatment of polycystic kidney disease,” Current Opinion in Nephrology and Hypertension, vol. 18, no. 2, pp. 99-106, 2009). There are two different forms of polycystic kidney disease, the more common autosomal dominant polycystic kidney disease (ADPKD), and the less common autosomal recessive polycystic kidney disease (ARPKD). The symptoms of PKD include aneurysms, flank pain, hematuria, renal colic, urinary tract infections, and hypertension. Currently treatment is limited to cyst aspiration, dialysis, and organ transplantation. There is an unmet medical need for the discovery and development of new therapies for treating cystic diseases, including PKD.

### SUMMARY OF THE DISCLOSURE

It has been discovered that modulators of peroxisome proliferator-activated receptors (PPARs), and in particular, PPARγ, are useful for treating cystic diseases, including PKD and ADPKD. PPARγ modulators, and in particular PPARγ agonists, are insulin-sensitizing agents and are commercially available for the treatment of diabetes. Based on cell culture models of the principal cell type of the distal nephron, PPARγ agonists inhibit cAMP-stimulated anion transport via cystic fibrosis transmembrane regulator (CFTR) by inhibiting the synthesis of the CFTR protein. Through inhibitor and electrophysiological studies it has been shown that CFTR is the Cl⁻ channel responsible for the cyst growth in PKD (Davidow C. J. et al., “The cystic fibrosis transmembrane conductance regulator mediates transepithelial fluid secretion by human autosomal dominant polycystic kidney disease epithelium in vitro,” Kidney International, vol. 50, no. 1, pp. 208-218, 1996; Muchatuta M. N. et al., “Structural and functional analysis of liver cysts from BALB/c-cpk mouse model of polycystic kidney disease,” Experimental Biology and Medicine, vol. 234, no. 1, pp. 17-27, 2009). Thus, it is believed herein that compounds capable of inhibiting CFTR synthesis are useful for treating cystic diseases, including PKD and ADPKD. However, PPARγ modulators, and in particular PPARγ agonists, are generally administered at relatively high doses, and have been observed to cause undesirable side effects. It has also been surprisingly discovered herein that low doses of PPARγ modulators, including those doses that are not clinically effective or efficacious in treating diabetes, are highly efficacious in treating cystic diseases, including PKD and ADPKD. Without being bound by theory, it is believed herein that the efficacy of compounds described is due at least in part to theory activity as CFTR synthesis inhibitors. It is appreciated herein that such low doses may be advantageous in order to avoid side effects of PPARγ modulators, including PPARγ agonists that have been observed at conventional doses used in treating diabetes.

Generally, methods, uses, compositions, and unit dosage forms are described herein for treating a host animal having a cystic disease, such as PKD or ADPKD. The methods include the step of administering to the host animal a therapeutically effective amount of a PPARγ modulator for treating the cystic disease. In one aspect, the therapeutically effective amount is that amount that is less than the clinically effective amount of the PPARγ modulator used as a monotherapy for treating diabetes.

In another aspect, uses of PPARγ modulator in the manufacture of medicaments for treating the cystic diseases, including PKD and ADPKD, are described herein. In one aspect, the medicaments include a therapeutically effective amount of a PPARγ modulator for treating the cystic disease. In another aspect, the therapeutically effective amount is that amount that is less than the clinically effective amount of the PPARγ modulator used as a monotherapy for treating diabetes.

In yet another, pharmaceutical compositions that include a therapeutically effective amount of one or more PPARγ modulators for treating cystic diseases, including PKD and ADPKD, are described herein. In one aspect, the therapeutically effective amount is that amount that is less than the clinically effective amount of the PPARγ modulator used as a monotherapy for treating diabetes. In another embodiment, unit doses and unit dosage forms that include a therapeutically effective amount of one or more PPARγ modulators for treating cystic diseases, including PKD and ADPKD, are described herein. In one aspect, the therapeutically effective amount is that amount that is less than the clinically effective amount of the PPARγ modulator used as a monotherapy for treating diabetes.

Accordingly, in one illustrative embodiment, the present disclosure is directed to a method for treating a host animal having a cystic disease. The method comprises the step of administering to the host animal a therapeutically effective amount of one or more PPARγ modulators selected from the group consisting of muraglitazar (CAS-No. 331741-94-7), pioglitazone (CAS-No. 111025-46-8), farglitazar (CAS-No. 196808-45-4), naveglitazar (CAS-No. 476436-68-7), netoglitazone (CAS-NO. 161600-01-7), rivoglitazone (CAS-No. 185428-18-6), sodelglitazar (GW-677954; CAS-No. 622402-24-8), troglitazone, tesaglitazar, ragaglitazar, isohumulone, (−)-Halofenate (CAS-No. 024136-23-0), K-111 (CAS-No. 221564-97-2), GW-677954, GW7845, L-796449, and DJ5 for treating the cystic disease, where the therapeutically effective amount is less than the clinically effective amount of the one or more PPARγ modulators as a monotherapy for treating diabetes.

In another illustrative embodiment, the present disclosure is directed to a method for treating a host animal having a cystic disease. The method comprises the step of administering to the host animal a therapeutically effective amount of pioglitazone for treating the cystic disease. The therapeutically effective amount is from about 1 μg/kg to about 200 μg/kg total body weight.

In yet another illustrative embodiment, the present disclosure is directed to a unit dose or unit dosage form comprising a therapeutically effective amount of pioglitazone for treating a cystic disease in an adult host animal. The therapeutically effective amount is from about 0.1 mg to about 15 mg.

It is appreciated herein that the compounds described herein may be used alone or in combination with other compounds useful for treating cystic diseases, including those compounds that may be therapeutically effective by the same or different modes of action. In addition, it is appreciated herein that the compounds described herein may be used in combination with other compounds that are administered to treat other symptoms of cystic diseases.

### BRIEF DESCRIPTION OF THE DRAWINGS

FIGS. 1A-1E show the effect of a 24 week treatment method with rosiglitazone on the PCK rat, a model of PKD. Particularly, FIG. 1A shows the effect of the 24 week treatment on body weight. FIG. 1B shows the effect of the 24 week treatment on kidney weight. FIG. 1C shows kidney weight as a percentage of body weight. FIG. 1D shows renal cyst size as percentage of kidney volume. FIG. 1E shows the effect of the 24 week treatment on renal cyst volume. Animals were fed rosliglitazone at the concentrations listed starting at the time of weaning (4 weeks) to 28 weeks of age. Data are plotted as means±SEM. Statistics were completed using Prostat, using one-tailed ANOVA, where a p-value<0.05 was considered significant. Further statistics were completed using the ANOVA F-test, in which the null hypothesis of the control vs. rosiglitazone treated was equal, if rejected, the data was significant.

FIGS. 2A-2C show histological cross-sections of the kidney from the PCK rats after 24 week treatment with rosiglitazone. FIG. 2A: 0 mg/kg BW or control, FIG. 2B: 4.0 mg/kg BW or high, FIG. 2C: 0.04 mg/kg BW or low.

FIGS. 3A-3C show the effect of a 13 day treatment method with pioglitazone on the W-WPK rat. Particularly, FIG. 3A shows the effect of the 13 day treatment on body weight. FIG. 3B shows the effect of the 13 day treatment on kidney weight. FIG. 3C shows kidney weight as a percentage of body weight. Animals were fed pioglitazone at the concentrations listed starting at the age of day 5. Data are plotted as means±SEM. Statistics were completed using Prostat, using one-tailed ANOVA, where a p-value<0.05 was considered significant. Further statistics were completed using the ANOVA F-test, in which the null hypothesis of the control vs. pioglitazone treated were equal, if was rejected, the data was significant.

### DETAILED DESCRIPTION OF THE DISCLOSURE

The present disclosure is directed to methods, uses, compositions, and unit dosage forms for treating a host animal having a cystic disease, such as PKD or ADPKD. More particularly, the disclosure is directed to the use of one or more PPARγ modulators, and in particular, PPARγ agonists, for treating the cystic disease.

As used herein, the terms “PPARγ modulator” and “PPARγ agonist” generally refer to the compounds that are capable of modulating or agonizing, respectively, the activity of PPARγ either in vitro or in vivo, including the compounds described herein and analogs and derivatives thereof.

It is to be understood that such derivatives may include prodrugs of the PPARγ modulators, PPARγ agonists, and compounds described herein that include one or more protection or protecting groups, including compounds that are used in the preparation of other compounds described herein.

Illustrative PPARγ agonists for use in the methods, medicaments, pharmaceutical compositions, unit doses, and unit dosage forms described herein include, but are not limited to, muraglitazar (CAS-No. 331741-94-7), rosiglitazone (CAS-NO. 122320-73-4), pioglitazone (CAS-No. 111025-46-8), farglitazar (CAS-No. 196808-45-4), naveglitazar (CAS-No. 476436-68-7), netoglitazone (CAS-NO. 161600-01-7), rivoglitazone (CAS-No. 185428-18-6), sodelglitazar (GW-677954; CAS-No. 622402-24-8), troglitazone, tesaglitazar, ragaglitazar, isohumulone, (−)-Halofenate (CAS-No. 024136-23-0), K-111 (CAS-No. 221564-97-2), GW-677954, GW7845, L-796449, and DJ5 and the like.

In another embodiment, the PPARγ modulator included in the methods, medicaments, pharmaceutical compositions, unit doses, and unit dosage forms described herein is selected from muraglitazar, rosiglitazone, pioglitazone, farglitazar, naveglitazar, netoglitazone, rivoglitazone, and sodelglitazar.

In another embodiment, the PPARγ modulator included in the methods, medicaments, pharmaceutical compositions, unit doses, and unit dosage forms described herein is selected from rosiglitazone, pioglitazone, and farglitazar.

It has also been discovered herein that the relative potency of PPARγ agonists at PPARγ is parallel to the potency of those compounds at the CTFR, which supports the conclusion that PPARγ modulators, including PPARγ agonists, will generally also be useful in the methods, medicaments, pharmaceutical compositions, unit doses, and unit dosage forms described herein for treating cystic diseases.

In another embodiment of the methods, medicaments, pharmaceutical compositions, unit doses, and unit dosage forms described herein, the one or more PPARγ modulators, such as PPARγ agonists, are administered at low doses.

As used herein, “low dose” refers to a therapeutically effective amount of the one or more PPARγ modulators that is less than the clinically effective amount of the one or more PPARγ modulators as a monotherapy for treating diabetes as known in the art. For example, the PPARγ modulators may be used in a therapeutically effective amount that is about 75% or less than the clinically effective amount of the one or more PPARγ modulators as a monotherapy for treating diabetes, including about 50% or less than the clinically effective amount, including about 25% or less than the clinically effective amount, including about 10% or less than the clinically effective amount, including about 5% or less than the clinically effective amount, and including about 1% or less than the clinically effective amount of the one or more PPARγ modulators.

By way of example, in one illustrative embodiment, at least one of the PPARγ modulators is pioglitazone, and the pioglitazone is administered in a therapeutically effective amount between about 1 μg/kg and about 200 μg/kg total body weight, including between about 1 μg/kg and about 100 μg/kg total body weight, and including between about 1 μg/kg and about 50 μg/kg total body weight, and including between about 1 μg/kg and about 25 μg/kg total body weight, and including between about 5 μg/kg and about 200 μg/kg total body weight, and including between about 5 μg/kg and about 100 μg/kg total body weight, and including between about 5 μg/kg and about 50 μg/kg total body weight, and including between about 5 μg/kg and about 25 μg/kg total body weight, and including between about 10 μg/kg and about 200 μg/kg total body weight, and including between about 10 μg/kg and about 100 μg/kg total body weight, and including between about 10 μg/kg and about 50 μg/kg total body weight, and including between about 10 μg/kg and about 25 μg/kg total body weight, and including between about 50 μg/kg and about 200 μg/kg total body weight, and including between about 50 μg/kg and about 100 μg/kg total body weight.

In another illustrative embodiment, at least one of the PPARγ modulators is rosiglitazone, and the therapeutically effective amount is between about 0.1 μg/kg and about 50 μg/kg total body weight, including between about 0.1 μg/kg and about 25 μg/kg total body weight, and including between about 0.1 μg/kg and about 10 μg/kg total body weight, and including between about 0.1 μg/kg and about 5 μg/kg total body weight, and including between about 0.5 μg/kg and about 50 μg/kg total body weight, and including between about 0.5 μg/kg and about 25 μg/kg total body weight, and including between about 0.5 μg/kg and about 10 μg/kg total body weight, and including between about 0.5 μg/kg and about 5 μg/kg total body weight, and including between about 1 μg/kg and about 50 μg/kg total body weight, and including between about 1 μg/kg and about 25 μg/kg total body weight, and including between about 1 μg/kg and about 10 μg/kg total body weight, and including between about 1 μg/kg and about 5 μg/kg total body weight.

In yet another illustrative embodiment, at least one of the PPARγ modulators is farglitazar, and the therapeutically effective amount is between about 0.1 μg/kg and about 50 μg/kg total body weight, including between about 0.1 μg/kg and about 25 μg/kg total body weight, and including between about 0.1 μg/kg and about 10 μg/kg total body weight, and including between about 0.1 μg/kg and about 5 μg/kg total body weight, and including between about 0.5 μg/kg and about 50 μg/kg total body weight, and including between about 0.5 μg/kg and about 25 μg/kg total body weight, and including between about 0.5 μg/kg and about 10 μg/kg total body weight, and including between about 0.5 μg/kg and about 5 μg/kg total body weight, and including between about 1 μg/kg and about 50 μg/kg total body weight, and including between about 1 μg/kg and about 25 μg/kg total body weight, and including between about 1 μg/kg and about 10 μg/kg total body weight, and including between about 1 μg/kg and about 5 μg/kg total body weight.

When the PPARγ modulator is used in a unit dose or unit dosage form, it should be understood that the unit dose or unit dosage form can be in a single or divided form as discussed more fully below. Typically, the unit dose or unit dosage form will include a therapeutically effective amount of one or more PPARγ modulators for treating a cystic disease in a host animal, where the therapeutically effective amount is less than the clinically effective amount of the one or more PPARγ modulators for treating diabetes. By way of example, the unit dose or unit dosage form includes a therapeutically effective amount of one or more PPARγ modulators that is 75% of the clinically effective amount of the one or more PPARγ modulators for treating diabetes as known in the art, including a therapeutically effective amount that is 50% of the clinically effective amount, and including a therapeutically effective amount that is 25% of the clinically effective amount, and including a therapeutically effective amount that is 10% of the clinically effective amount, and including a therapeutically effective amount that is 5% of the clinically effective amount, and including a therapeutically effective amount is 1% of the clinically effective amount.

In one embodiment, the unit dose or unit dosage form is administered to an adult human animal for treating a cystic disease. As used herein, an “adult” refers to a subject that is over the age of 18 years. In another embodiment, the unit dose or unit dosage form is administered to a pediatric human. As used herein, a “pediatric” refers to a subject under the age of 18 years.

By way of example, in one illustrative embodiment, the unit dose or unit dosage form includes pioglitazone as at least one of the PPARγ modulators, and the unit dose or unit dosage includes pioglitazone in a therapeutically effective amount of between about 0.1 mg and about 15 mg, including between about 0.1 mg and about 10 mg, and including between about 0.1 mg and about 5 mg, and including between about 0.5 mg and about 15 mg, and including between about 0.5 mg and about 10 mg, and including about 0.5 mg and about 5 mg, and including between about 1 mg and about 15 mg, and including between about 1 mg and about 10 mg, and including between about 1 mg and about 5 mg, and including between about 5 mg and about 15 mg, and including between about 5 mg and about 10 mg.

In another illustrative embodiment, the unit dose or unit dosage form includes rosiglitazone as at least one of the PPARγ modulators, and the unit dose or unit dosage includes rosiglitazone in a therapeutically effective amount of between about 0.01 mg and about 4 mg, including between about 0.01 mg and about 2 mg, and including between about 0.01 mg and about 1 mg, and including between about 0.05 mg and about 4 mg, and including between about 0.05 mg and about 2 mg, and including between about 0.05 mg and about 1 mg, and including between about 0.1 mg and about 4 mg, and including between about 0.1 mg and about 2 mg, and including between about 0.1 mg and about 1 mg, and including between about 0.5 mg and about 4 mg, and including between about 0.5 mg and about 2 mg, and including between about 0.5 mg and about 1 mg, and including between about 0.01 mg and about 2 mg.

In another illustrative embodiment, the unit dose or unit dosage form includes farglitazar as at least one of the PPARγ modulators, and the unit dose or unit dosage includes farglitazar in a therapeutically effective amount of between about 0.01 mg and about 5 mg, including between about 0.01 mg and about 2 mg, and including between about 0.01 mg and about 1 mg, and including between about 0.01 mg and about 0.5 mg, and including between about 0.05 mg and about 5 mg, and including between about 0.05 mg and about 2 mg, and including between about 0.05 mg and about 1 mg, and including between about 0.05 mg and about 0.5 mg, and including between about 0.1 mg and about 5 mg, and including between about 0.1 mg and about 2 mg, and including between about 0.1 mg and about 1 mg, and including between about 0.1 mg and about 0.5 mg, and including between about 0.5 mg and about 5 mg, and including between about 0.5 mg and about 2 mg, and including between about 0.5 mg and about 1 mg, and including between about 0.05 mg and about 0.2 mg, and including between about 0.05 mg and about 0.1 mg.

In each of the foregoing and following embodiments, it is to be understood that the compounds and formulae include and represent not only all pharmaceutically acceptable salts of the compounds, but also include any and all hydrates and/or solvates of the compound formulae. It is appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent any and all crystalline forms, partially crystalline forms, and non-crystalline and/or amorphous forms of the compounds.

The term “prodrug” as used herein generally refers to any compound that when administered to a biological system generates a biologically active compound as a result of one or more spontaneous chemical reaction(s), enzyme-catalyzed chemical reaction(s), and/or metabolic chemical reaction(s), or a combination thereof. In vivo, the prodrug is typically acted upon by an enzyme (such as esterases, amidases, phosphatases, and the like), simple biological chemistry, or other process in vivo to liberate or regenerate the more pharmacologically active drug. This activation may occur through the action of an endogenous host enzyme or a non-endogenous enzyme that is administered to the host preceding, following, or during administration of the prodrug. Additional details of prodrug use are described in U.S. Pat. No. 5,627,165; and Pathalk et al., “Enzymic protecting group techniques in organic synthesis,” Stereosel. Biocatal. 775-797 (2000). It is appreciated that the prodrug is advantageously converted to the original drug as soon as the goal, such as targeted delivery, safety, stability, and the like is achieved, followed by the subsequent rapid elimination of the released remains of the group forming the prodrug.

Prodrugs may be prepared from the compounds described herein by attaching groups that ultimately cleave in vivo to one or more functional groups present on the compound, such as —OH—, —SH, —CO₂H, —NR₂. Illustrative prodrugs include but are not limited to carboxylate esters where the group is alkyl, aryl, arylalkyl, heteroaryl, heteroarylalkyl, acyloxyalkyl, alkoxycarbonyloxyalkyl as well as esters of hydroxyl, thiol and amines where the group attached is an acyl group, an alkoxycarbonyl, aminocarbonyl, phosphate or sulfate. Illustrative esters, also referred to as active esters, include but are not limited to 1-indanyl, N-oxysuccinimide; acyloxyalkyl groups such as acetoxymethyl, pivaloyloxymethyl, β-acetoxyethyl, β-pivaloyloxyethyl, 1-(cyclohexylcarbonyloxy)prop-1-yl, (1-aminoethyl)carbonyloxymethyl, and the like; alkoxycarbonyloxyalkyl groups, such as ethoxycarbonyloxymethyl, α-ethoxycarbonyloxyethyl, β-ethoxycarbonyloxyethyl, and the like; dialkylaminoalkyl groups, including di-lower alkylamino alkyl groups, such as dimethylaminomethyl, dimethylaminoethyl, diethylaminomethyl, diethylaminoethyl, and the like; 2-(alkoxycarbonyl)-2-alkenyl groups such as 2-(isobutoxycarbonyl) pent-2-enyl, 2-(ethoxycarbonyl)but-2-enyl, and the like; and lactone groups such as phthalidyl, dimethoxyphthalidyl, and the like.

Further illustrative prodrugs contain a chemical moiety, such as an amide or phosphorus group functioning to increase solubility and/or stability of the compounds described herein. Further illustrative prodrugs for amino groups include, but are not limited to, (C₃-C₂₀)alkanoyl; halo-(C₃-C₂₀)alkanoyl; (C₃-C₂₀)alkenoyl; (C₄-C₇)cycloalkanoyl; (C₃-C₆)-cycloalkyl(C₂-C₁₆)alkanoyl; optionally substituted aroyl, such as unsubstituted aroyl or aroyl substituted by 1 to 3 substituents selected from the group consisting of halogen, cyano, trifluoromethanesulphonyloxy, (C₁-C₃)alkyl and (C₁-C₃)alkoxy, each of which is optionally further substituted with one or more of 1 to 3 halogen atoms; optionally substituted aryl(C₂-C₁₆)alkanoyl and optionally substituted heteroaryl(C₂-C₁₆)alkanoyl, such as the aryl or heteroaryl radical being unsubstituted or substituted by 1 to 3 substituents selected from the group consisting of halogen, (C₁-C₃)alkyl and (C₁-C₃)alkoxy, each of which is optionally further substituted with 1 to 3 halogen atoms; and optionally substituted heteroarylalkanoyl having one to three heteroatoms selected from O, S and N in the heteroaryl moiety and 2 to 10 carbon atoms in the alkanoyl moiety, such as the heteroaryl radical being unsubstituted or substituted by 1 to 3 substituents selected from the group consisting of halogen, cyano, trifluoromethanesulphonyloxy, (C₁-C₃)alkyl, and (C₁-C₃)alkoxy, each of which is optionally further substituted with 1 to 3 halogen atoms. The groups illustrated are exemplary, not exhaustive, and may be prepared by conventional processes.

It is understood that the prodrugs themselves may not possess significant biological activity, but instead undergo one or more spontaneous chemical reaction(s), enzyme-catalyzed chemical reaction(s), and/or metabolic chemical reaction(s), or a combination thereof after administration in vivo to produce the compound described herein that is biologically active or is a precursor of the biologically active compound. However, it is appreciated that in some cases, the prodrug is biologically active. It is also appreciated that prodrugs may often serve to improve drug efficacy or safety through improved oral bioavailability, pharmacodynamic half-life, and the like. Prodrugs also refer to derivatives of the compounds described herein that include groups that simply mask undesirable drug properties or improve drug delivery. For example, one or more compounds described herein may exhibit an undesirable property that is advantageously blocked or minimized, may become pharmacological, pharmaceutical, or pharmacokinetic barriers in clinical drug application, such as low oral drug absorption, lack of site specificity, chemical instability, toxicity, and poor patient acceptance (bad taste, odor, pain at injection site, and the like), and others. It is appreciated herein that a prodrug, or other strategy using reversible derivatives, can be useful in the optimization of the clinical application of a drug.

As used herein, the term “composition” generally refers to any product comprising the specified ingredients in the specified amounts, as well as any product which results, directly or indirectly, from combinations of the specified ingredients in the specified amounts. It is to be understood that the compositions described herein may be prepared from isolated compounds described herein or from salts, solutions, hydrates, solvates, and other forms of the compounds described herein. It is also to be understood that the compositions may be prepared from various amorphous, non-amorphous, partially crystalline, crystalline, and/or other morphological forms of the compounds described herein. It is also to be understood that the compositions may be prepared from various hydrates and/or solvates of the compounds described herein. Accordingly, such pharmaceutical compositions that recite compounds described herein are to be understood to include each of, or any combination of, the various morphological forms and/or solvate or hydrate forms of the compounds described herein. Illustratively, compositions may include one or more carriers, diluents, and/or excipients. The compounds described herein, or compositions containing them, may be formulated in a therapeutically effective amount in any conventional dosage forms appropriate for the methods described herein. The compounds described herein, or compositions containing them, including such formulations, may be administered by a wide variety of conventional routes for the methods described herein, and in a wide variety of dosage formats, utilizing known procedures (see generally, Remington: The Science and Practice of Pharmacy, (21^{st} ed., 2005)).

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

It is also appreciated that the therapeutically effective amount, whether referring to monotherapy or combination therapy, is advantageously selected with reference to any toxicity, or other undesirable side effect, that might occur during administration of one or more of the compounds described herein. Further, it is appreciated that the co-therapies described herein may allow for the administration of lower doses of compounds that show such toxicity, or other undesirable side effect, where those lower doses are below thresholds of toxicity or lower in the therapeutic window than would otherwise be administered in the absence of a cotherapy.

The term “clinically effective amount” as used herein, generally refers to that amount of active compound or pharmaceutical agent that provides a clinically relevant improved outcome in treating the disease. For example, a clinically effective amount may be, and is often, higher than a therapeutically effective amount because the attending physician will seek to maximize the therapeutic effect rather than administer compound at the threshold amount that would lead to a biological or medicinal response in the tissue system, animal or human that is being sought by a researcher, veterinarian, medical doctor or other clinician, which includes alleviation of the symptoms of the disease or disorder being treated.

In addition to the illustrative dosages and dosing protocols described herein, it is to be understood that an effective amount of any one or a mixture of the compounds described herein can be readily determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “administering” as used herein includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically-acceptable carriers, adjuvants, and vehicles.

Illustrative formats for oral administration include tablets, capsules, elixirs, syrups, and the like.

Illustrative routes for parenteral administration include intravenous, intraarterial, intraperitoneal, epidurial, intraurethral, intrasternal, intramuscular and subcutaneous, as well as any other art recognized route of parenteral administration.

Illustratively, administering includes local use, such as when administered locally to the site of disease, injury, or defect, or to a particular organ or tissue system. Illustrative local administration may be performed during open surgery, or other procedures when the site of disease, injury, or defect is accessible. Alternatively, local administration may be performed using parenteral delivery where the compound or compositions described herein are deposited locally to the site without general distribution to multiple other non-target sites in the patient being treated. It is further appreciated that local administration may be directly in the injury site, or locally in the surrounding tissue. Similar variations regarding local delivery to particular tissue types, such as organs, and the like, are also described herein. Illustratively, compounds may be administered directly to the nervous system including, but not limited to, intracerebral, intraventricular, intracerebroventricular, intrathecal, intracisternal, intraspinal and/or peri-spinal routes of administration by delivery via intracranial or intravertebral needles and/or catheters with or without pump devices.

Depending upon the disease as described herein, the route of administration and/or whether the compounds and/or compositions are administered locally or systemically, a wide range of permissible dosages are contemplated herein, including doses falling in the range from about 1 μg/kg to about 1 g/kg. The dosages may be single or divided, and may be administered according to a wide variety of protocols, including q.d., b.i.d., t.i.d., or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In making the pharmaceutical compositions of the compounds described herein, a therapeutically effective amount of one or more compounds in any of the various forms described herein may be mixed with one or more excipients, diluted by one or more excipients, or enclosed within such a carrier which can be in the form of a capsule, sachet, paper, or other container. Excipients may serve as a diluent, and can be solid, semi-solid, or liquid materials, which act as a vehicle, carrier or medium for the active ingredient. Thus, the formulation compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders. The compositions may contain anywhere from about 0.1% to about 99.9% active ingredients, depending upon the selected dose and dosage form.

The effective use of the compounds, compositions, and methods described herein for treating or ameliorating one or more effects of cystic diseases using one or more compounds described herein may be based upon animal models, such as murine, canine, porcine, and non-human primate animal models of disease. For example, it is understood that cystic diseases, including PKD and ADPKD, in humans may be characterized by a loss of function, and/or the development of symptoms, each of which may be elicited in animals, such as mice, and other surrogate test animals. In particular the PCK rat model, and the W-WPK rat model, may be used to evaluate the methods of treatment and the pharmaceutical compositions described herein to determine the therapeutically effective amounts described herein.

The following examples further illustrate specific embodiments of the invention; however, the following illustrative examples should not be interpreted in any way to limit the invention.

### EXAMPLES

### Example

PCK rat animal model of slowly-progressing PKD. The PCK rat is an animal model that has a orthologous genetic mutation to the human autosomal recessive polycystic kidney disease (ARPKD), but has the phenotypic characteristics of human ADPKD, including renal and liver fibrocystic disease. The Pck gene of the rat is an orthologue to the PKHD-1 gene responsible for ARPKD. The animal model develops both kidney and liver fibrocystic diseases, and is slow progressing. There is also a gender dimorphism of the rat model; females develop more severe liver disease while males develop more severe kidney disease, which resembles what is seen in human ADPKD (Mason S. B. et al., “Disease Stage Characterization of Hepatorenal Fibrocystic Pathology in the PCK Rat Model of ARPKD,” The Anatomical Record, vol. 293, pp. 1279-1288, 2010). Only females are used in the study.

EXAMPLE. W-WPK rat animal model of rapidly-progressing PKD. The W-WPK rat model is a rapidly progressing model that shows full development of the disease at the age of 21 days (Gattone V. H. et al., “Development of Multiorgan Patholog in the wpk Rat Model of Polycystic Kidney Disease,” The Anatomical Record, vol. 277, pp. 384-395, 2004). The mutant gene is the MKS3 gene in humans and MKS3 gene in rats. Meckelin is the protein associated with the gene mutation, which has 995 amino acids in humans and 997 amino acids in the rat. The rat and human protein are 84% identical and 91% similar (Smith U. M. et al., “The transmembrane protein meckelin (MKS3) is mutated in Meckel-Gruber syndrome and the wpk rat,” Nature Genetics, vol. 28, no. 2, pp. 191-96, 2006). The W-WPK rat was reportedly developed for its renal cystic disease, but it has demonstrated phenotypic CNS malformations, such as hypoplasia, agenesis of the corpus callosum, and severe hydrocephalus. The model is used for both renal cystic disease and Meckel-Gruber Syndrome (Gattone V. H. et al., 2004).

### Example

Low dose efficacy of PPARγ modulators in cyst growth inhibition in the PCK rat model of PKD. Briefly, after weaning, test animals are fed a diet containing increasing doses of a PPARγ modulator, normalized to body weight, and compared to untreated control fed normal diet. A hematocrit is measured to assess the presence or absence of fluid retention. Rosiglitazone (4, 0.4, 0.04 mg/kg BW) showed that the lowest dose (0.04 mg/kg BW) was effective in a 24 week feeding study. The untreated control group fed the same diet without any PPARγ modulators. PPARγ modulators significantly slow renal cyst growth, and the low dose showed a decreased number or severity of undesirable side effects, without a loss of efficacy.

Animals are sacrificed and total body weight is recorded. Total kidney weight is recorded, total liver weight is recorded, and heart weight is recorded. Blood is collected via cardiac puncture, and serum and plasma samples are collected. The liver and kidney were embedded in paraffin and transversely sectioned and stained with hematoxylin, and eosin. The percent kidney weight to body weight was calculated. After histology on the kidney, renal cyst volume was determined through point count stereology methods. From the total liver weight, the percent liver to body weight was calculated. Following histology and picrosiruis red staining, the percent fibrosis can be determined through point count stereology methods. Heart weight is determined and the percent heart weight to body weight is calculated to assess heart disease, such as due to fluid retention, a reported side effect associated with PPARγ agonist treatment. Hearts are preserved in 4% paraformaldehyde. Liver enzymes, glucose levels, and ion levels are evaluated using the blood and urine samples.

High dose, but not low rosiglitazone significantly increased body weight. Without being bound by theory, it is believed herein this undesirable side effect is caused by fluid retention or adipogenesis. Such side effects have been reported, and are established to be due to high dose PPARγ agonist therapy. Both high and low dose significantly decreased kidney weight as a percent of body weight, total kidney volume and renal cyst volume. The intermediate dose, 0.4 mg/kg BW, is not shown. It should be noted that the high and low doses differ by two orders of magnitude (100×). Statistical significance was not observed between any of the treatment groups at the high, intermediate, and low doses (4, 0.4, and 0.04 mg/kg BW) for the efficacious endpoints of renal cysts as a percentage of kidney volume and renal cyst volume, indicating that even the lowest doses evaluated are efficacious.

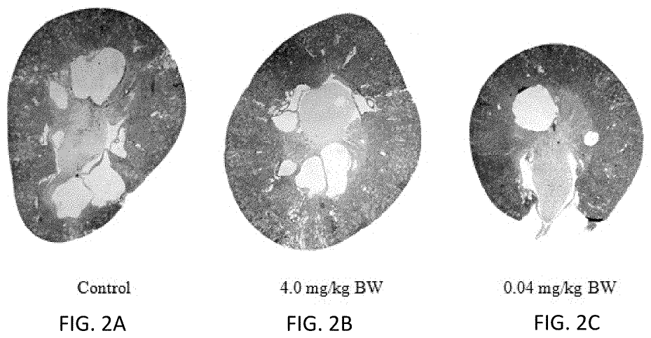

Transverse renal sections from 28 week old PCK rats are taken after the animal has been fed the PPARγ modulator, such as rosiglitazone, for 24 weeks. As shown in the kidney sections in FIGS. 2A-2C, the renal cystic burden in the treated rats is significantly decreased. Heart weight and hematocrits are not different from the control animals compared to the animals treated with low dose PPARγ modulators, such as rosiglitazone. The conventional (high) doses of PPARγ modulators, such as rosiglitazone, leads to additional undesired side effects. In particular, 9/12 test animals in the high dose group died or had to be sacrificed during the treatment period due to the development of cholangitis.

The low dose (100 fold lower than the high dose) was as efficacious in reducing all parameters of cystic growth as the high dose with no detected side effects (increased body weight or cholangitis).

Serum electrolytes are not altered by the low dose PPARγ modulator, such as rosiglitazone, treatment at any concentration. Hematocrits were unchanged by any of the three concentrations of drugs (data not shown).

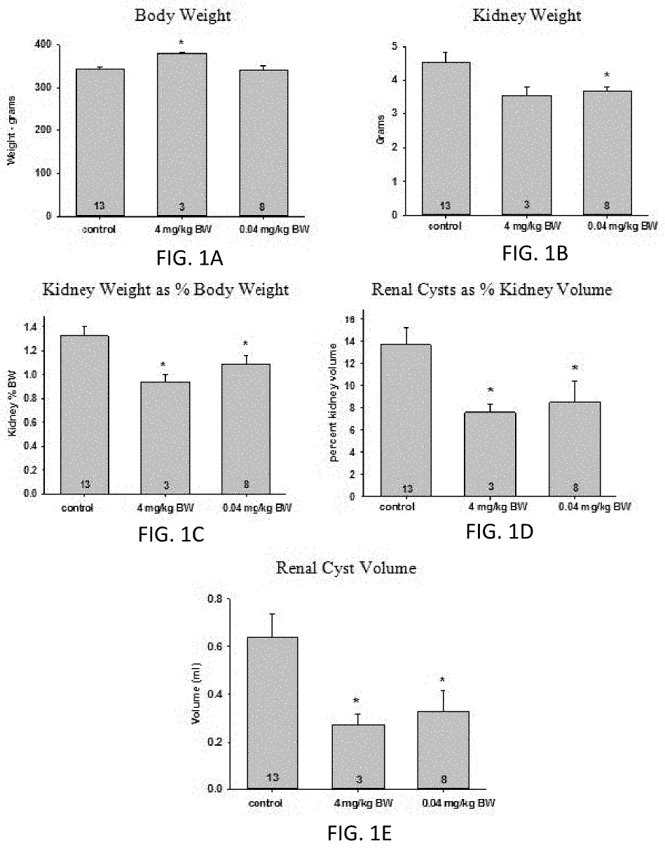

A significant change in glucose or hematocrit of the animals at any concentration was not observed indicating that the insulin sensitizing drug did not cause hypoglycemia and did not cause severe fluid retention. However, as shown in FIG. 1A, the body weight of the high dose treated animals did significantly increase compared to the control, which could be due to fluid retention or adipogenesis, known side effects of the PPARγ agonists. Without being bound by theory, it is believed herein that the normal hematocrits suggest that the body weight gain may be due in part to fluid retention which is not severe enough to cause a change in hematocrit.

TABLE 1
24 week treatment with rosiglitazone in PCK model.

| | 24 Week Diet | | |
|---|---|---|---|
| | Control | Rosiglitazone | |
| | Diet | 4.0 mg/kg BW | |
| | n = 12 | n = 3 | Statistics (a) |
| Body Weight (BW) g | 340.68 ± 5.55 | 378.5 ± 2.47 | S |
| Kidney Weight (KW) g | 4.33 ± 0.18 | 3.56 ± 0.26 | S |
| KW % BW | 1.22 ± 0.04 | 0.94 ± 0.06 | S |
| % Cyst Volume | 12.70 ± 1.32 | 7.58 ± 0.72 | S |
| Renal Cyst Volume (mL) | 0.53 ± 0.06 | 0.27 ± 0.05 | S |
| Liver Weight (LW) g | 23.11 ± 1.44 | 22.66 ± 5.39 | NS |
| LW % BW | 6.77 ± 0.43 | 5.97 ± 1.38 | NS |
| Liver % Fibrocystic | 31.10 ± 2.02 | 36.17 ± 0.38 | NS |
| Heart Weight (HW) | 1.29 ± 0.03 | 1.25 | Ns |

| | 24 Week Diet | | | | |
|---|---|---|---|---|---|
| | | Rosiglitazone | | Rosiglitazone | |
| | Control | 0.4 mg/kg | | 0.04 mg/kg | |
| | Diet only | BW | Statistics | BW | Statistics |
| | n = 12 | n = 8 | (a) | n = 8 | (a) |
| Body Weight (BW) g | 340.68 ± 5.55 | 338.94 ± 6.94 | NS | 342.11 ± 10.10 | NS |
| Kidney Weight | 4.33 ± 0.18 | 4.27 ± 0.22 | NS | 3.68 ± 0.14 | S |
| (KW) g | | | | | |
| KW % BW | 1.22 ± 0.04 | 1.27 ± 0.08 | NS | 0.95 ± 0.15 | S |
| % Cyst Volume | 12.70 ± 1.32 | 18.53 ± 2.55 | S | 8.49 ± 1.92 | S |
| Renal Cyst Volume | 0.53 ± 0.06 | 0.82 ± 0.14 | S | 0.33 ± 0.09 | S |
| (mL) | | | | | |
| Liver Weight (LW) g | 23.11 ± 1.44 | 22.05 ± 2.07 | NS | 24.23 ± 0.10 | NS |
| LW % BW | 6.77 ± 0.43 | 6.50 ± 0.57 | NS | 7.11 ± 0.27 | NS |
| Liver % Fibrocystic | 31.10 ± 2.02 | 36.81 ± 3.61 | NS | 35.36 ± 3.74 | NS |
| Heart Weight (HW) | 1.29 ± 0.03 | 1.15 ± 0.05 | NS | 1.23 ± 0.03 | NS |

(a) Statistical significance compared to control;
S = statistically significant;
NS = not statistically significant.

### Comparative Example

High dose PPARγ treatment. A reported study was performed using the PCK rat to determine the effect of oral feeding (7 or 14 weeks) of the PPARγ agonist, pioglitazone. PCK rats were either fed a control diet or a diet supplemented with pioglitazone. The 7 week study included concentrations of pioglitazone at 4 mg/kg BW and 20 mg/kg BW. The 14 week study included a concentration of pioglitazone at 20 mg/kg BW. The animals were sacrificed at the end of each 7 and 14 week study. In the 14 week study, blood was collected by cardiac puncture for serum analysis. The left kidney and right liver lobe were each collected and frozen in liquid nitrogen. The remaining kidney and liver lobe were each fixed, then removed and kept in 4% paraformaldehyde. The kidney and liver were each later embedded in paraffin and transversely sectioned and stained with hematoxylin, and eosin. Cyst volume was calculated using point count stereology methods. Fibrosis was assessed on a 1-4 scale (1 was normal and 4 was severe) after completing a picrosirius red staining. Immunohistochemistry by light microscopy and immunocytochemistry by transmission electron microscopy were conducted to stain for the CFTR channel. At the end of the study it was shown that there was improvement in the renal and liver cyst burden due predominantly to decreased cyst size. There was a variation between the male and female rats used in the study. It was reported that after about 18 weeks of disease progression in the animal model, male renal cysts enlarged resulting in severe renal dysfunction, while females developed greater liver cyst volume. The serum analysis showed well-preserved kidney function, but liver enzymes increased indicating compromised hepatic function. A conclusive effect of pioglitazone on fibrosis of either the kidney or the liver was not reported because fibrosis is mild in early stages (e.g., 7 or 14 weeks) of the disease in this PCK rat model. The bile ducts showed a decrease in apical expression of CFTR by electron microscopy (Blazer-Yost B. L. et al., “Pioglitazone Attenuates Cystic Burden in the PCK Rodent Model of Polycystic Kidney Disease, “PPAR Research, doi:10.1155/2010/274376, 2010).

### Example

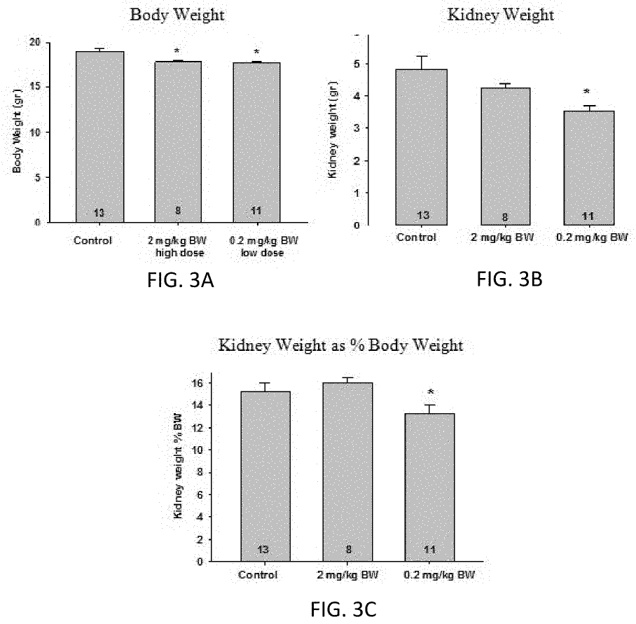

Low dose efficacy of PPARγ modulators in cyst growth inhibition in the W-WPK rat model of PKD. The W-WPK rat model has a maximal lifespan of 21 days; however, most affected animals become ill at or soon after about 18 days. Accordingly, the treatment period is set 5 days to 18 days. There were two treatments used, the high dose of 2 mg/kg BW pioglitazone, which is 10 fold lower than in the comparative example, and a low dose of 0.2 mg/kg BW. Test animals are sacrificed at day 18. FIGS. 3B and 3C surprisingly show that only the lowest dose of pioglitazone (0.2 mg/kg BW) was effective at decreasing the cystic burden whether measured as total kidney weight or as kidney weight as a percentage of body weight.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for treating a host animal having a cystic disease, the method comprising the step of administering to the host animal a therapeutically effective amount of one or more PPARγ modulators selected from the group consisting of muraglitazar (CAS-No. 331741-94-7), pioglitazone (CAS-No. 111025-46-8), farglitazar (CAS-No. 196808-45-4), naveglitazar (CAS-No. 476436-68-7), netoglitazone (CAS-NO. 161600-01-7), rivoglitazone (CAS-No. 185428-18-6), sodelglitazar (GW-677954; CAS-No. 622402-24-8), troglitazone, tesaglitazar, ragaglitazar, isohumulone, (−)-Halofenate (CAS-No. 024136-23-0), K-111 (CAS-No. 221564-97-2), GW-677954, GW7845, L-796449, and DJ5 for treating the cystic disease, wherein the therapeutically effective amount is less than the clinically effective amount of the one or more PPARγ modulators as a monotherapy for treating diabetes.

2. The method of claim 1 wherein the therapeutically effective amount is 75% or less of the clinically effective amount.

3. The method of claim 1 wherein the therapeutically effective amount is 50% or less of the clinically effective amount.

4. The method of claim 1 wherein the administering step includes oral administration.

5. The method of claim 1 wherein the cystic disease is a polycystic kidney disease.

6. A method for treating a host animal having a cystic disease, the method comprising the step of administering to the host animal a therapeutically effective amount of pioglitazone for treating the cystic disease, wherein the therapeutically effective amount is from about 1 μg/kg to about 200 μg/kg total body weight.

7. The method of claim 6 wherein the therapeutically effective amount is from about 1 μg/kg to about 100 μg/kg total body weight.

8. The method of claim 6 wherein the therapeutically effective amount is from about 1 μg/kg to about 50 μg/kg total body weight.

9. The method of claim 6 wherein the therapeutically effective amount is from about 1 μg/kg to about 25 μg/kg total body weight.

10. The method of claim 6 wherein the therapeutically effective amount is from about 5 μg/kg to about 200 μg/kg total body weight.

11. The method of claim 6 wherein the therapeutically effective amount is from about 10 μg/kg to about 100 μg/kg total body weight.

12. The method of claim 6 wherein the administering step includes oral administration.

13. The method of claim 6 wherein the administering step is performed b.i.d.

14. A unit dose or unit dosage form comprising a therapeutically effective amount of pioglitazone for treating a cystic disease in an adult host animal, wherein the therapeutically effective amount is from about 0.1 mg to about 15 mg.

15. The unit dose or unit dosage form of claim 14 wherein the therapeutically effective amount is from about 0.1 mg to about 10 mg.

16. The unit dose or unit dosage form of claim 14 wherein the therapeutically effective amount is from about 0.5 mg to about 15 mg.

17. The unit dose or unit dosage form of claim 14 wherein the therapeutically effective amount is from about 0.5 mg to about 10 mg.

18. The unit dose or unit dosage form of claim 14 wherein the therapeutically effective amount is from about 1 mg to about 15 mg.

19. The unit dose or unit dosage form of claim 14 being in single or divided form.

20. The unit dose or unit dosage form of claim 14 further comprising one or more carriers, diluents, or excipients, or a combination thereof.
